(19) United States
(12) Patent Application Publication
(10) Pub. No.: US 2019/0175123 A1
(43) Pub. Date: Jun. 13, 2019

(54) NUCLEAR MEDICINE DIAGNOSTIC APPARATUS FOR BREAST EXAMINATION AND DISTANCE ADJUSTMENT APPARATUS FOR NUCLEAR MEDICINE DIAGNOSTIC APPARATUS FOR BREAST EXAMINATION

(71) Applicant: Shimadzu Corporation, Kyoto (JP)

(72) Inventors: Atsushi OHTANI, Kyoto (JP); Kazushige TACHIBANA, Kyoto (JP)

(73) Assignee: Shimadzu Corporation, Kyoto (JP)

(21) Appl. No.: 16/039,498

(22) Filed: Jul. 19, 2018

(30) Foreign Application Priority Data
Dec. 13, 2017 (JP) 2017-238303

(51) Int. Cl. A61B 6/04; A61B 6/00

(52) U.S. Cl. CPC A61B 6/0435; A61B 6/4057; A61B 6/502; A61B 6/4208

(57) ABSTRACT

A nuclear medicine diagnostic apparatus for breast examination capable of diagnosing the entire breast of an examinee while the breast is in a free drooping state regardless of a drooping state of the examinee's breast, and a distance adjustment apparatus for the nuclear medicine diagnostic apparatus for breast examination to be used for the nuclear medicine diagnostic apparatus for breast examination are provided. When an examinee's breast does not fit within a radiation detection range of a radiation detection unit, a distance adjustment member including a first part, a second part and a third part is placed on a placement unit, and an examinee M is placed in a prone position on the distance adjustment member. Then, the breast of the examinee M is caused to droop to the radiation detection range in the radiation detection unit via an opening formed in the second part.

### FIELD

The present invention relates to a nuclear medicine diagnostic apparatus for breast examination for acquiring data for nuclear medicine based on radiation generated from a breast of an examinee to whom a radiopharmaceutical has been administered, and a distance adjustment apparatus for a nuclear medicine diagnostic apparatus for breast examination used for the nuclear medicine diagnostic apparatus for breast examination.

### BACKGROUND

In such a nuclear medicine diagnostic apparatus for breast examination, the breast of an examinee to whom a radiopharmaceutical has been administered in advance is inserted into a ring-shaped radiation detection unit in which a tunnel-like examination space is formed, and pair annihilation photons of, for example, 511 keV emitted from the breast of the examinee are detected by the radiation detection unit. Then, a time at which the photons are detected is measured, and when a difference between the times detected by two detectors in the radiation detection unit is within a certain time, the photons are counted as a pair of pair annihilation photons and, furthermore, specified as being at positions on a straight line of a pair of the detectors that detected a pair annihilation occurrence point. Emission data thus obtained is accumulated, and image reconstruction is performed using the accumulated emission data to acquire a PET image. Then, the PET image thus acquired is displayed on a monitor and transferred to a server in a hospital.

In such a nuclear medicine diagnostic apparatus for breast examination, two types of a method of positioning the radiation detection unit with respect to the breast of the examinee exist. One of them is a method in which the examinee is fixed at a predetermined position in a bed, a chair or the like and then the radiation detection unit is brought close to the examinee and positioned, and the other one is a method in which the radiation detection unit is fixed and the breast of the examinee is inserted into the radiation detection unit and positioned.

In Patent Literature 1, a table is disclosed for performing a medical procedure in which an examinee is placed on a top of a table where an opening for inserting the breast of the examinee is formed and the breast of the examinee is brought close to an X-ray imaging device with the breast of the examinee drooping through the opening and positioned. In the table described in this Patent Literature 1, an alternative insert having an opening of a larger or smaller diameter is selected and used, depending on a size of the breast of the examinee.

Patent Literature 1: JP-A-2016-185349

### SUMMARY

The ring-shaped radiation detection unit used in such a nuclear medicine diagnostic apparatus for breast examination has a detection range of radiation which is set as a certain region. For this reason, it is necessary to place the breast of the examinee within the detection range and to capture an image. At this time, in the case of adopting the method in which the radiation detection unit is brought close to the examinee and positioned as described above, even when a shape of the breast of the examinee is various, such a situation may be dealt with by moving the radiation detection unit. On the other hand, in the nuclear medicine diagnostic apparatus for breast examination which is placed in the detection range of radiation by the X-ray imaging apparatus with the breast of the examinee drooping, as described in Patent Literature 1, it is not possible to examine the entire breast due to drooping of the breast depending on a physique and an age of the examinee.

Therefore, in such a nuclear medicine diagnostic apparatus for breast examination, it may be considered to support the breast of the examinee from below when the breast falls outside the detection range. However, in such a configuration, the breast is deformed and it is not possible to carry out the examination while the breast is in a free drooping state. When the examination is performed while the breast is in the free drooping state, there are advantages that it is easy to understand a shape of the entire breast, an expansion of a mammary gland or the like based on a position of a nipple, and it is easy to compare a position of lesion with morphological information by an MRI and the like which photographs in the same prone position. Therefore, when it is not possible to perform the examination while the breast is in the free drooping state, the advantages described above cannot be acquired.

The present invention has been made to solve the above problems, and it is an object of the present invention to provide a nuclear medicine diagnostic apparatus for breast examination capable of diagnosing the entire breast of an examinee while the breast is in a free drooping state regardless of a drooping state of the examinee's breast, and a distance adjustment apparatus for the nuclear medicine diagnostic apparatus for breast examination to be used for the nuclear medicine diagnostic apparatus for breast examination.

The invention described in claim 1 is a nuclear medicine diagnostic apparatus for breast examination including a placement unit where an opening is formed and a radiation detecting unit disposed around an examinee's breast drooping from the opening of the placement unit in a state where the examinee is in a prone position on the placement unit, wherein the nuclear medicine diagnostic apparatus includes a distance adjustment member that has an opening connected to the opening of the placement unit when the distance adjustment member is placed on the placement unit, and changes a distance between the radiation detection unit and the examinee by being placed on the placement unit.

The invention described in claim 2 is that the distance adjustment member is divided into a plurality of parts with respect to a direction of a body axis of the examinee, in the invention described in claim 1.

The invention described in claim 3 includes a connection member that connects each part of the distance adjustment member to each other, in the invention described in claim 2.

The invention described in claim 4, one of the parts of the distance adjustment member is configured to place a head of the examinee thereon, and the part configured to place the head of the examinee thereon is engageable with a concave portion formed in the placement unit, in the invention described in claim 3.

The invention described in claim 5 is that an inner diameter of the opening formed in the distance adjustment member is larger than an inner diameter of the opening formed in the placement unit, in the invention described in any one of claims 1 to 4.

The invention described in claim 6 is an adjustment apparatus that is used in a nuclear medicine diagnostic apparatus for breast examination including a placement unit where an opening is formed and a radiation detecting unit disposed around an examinee's breast drooping from the opening of the placement unit in a state where the examinee is in a prone position on the placement unit, wherein the adjustment apparatus has an opening formed thereon and connected to the opening of the placement unit when the adjustment apparatus is placed on the placement unit, and changes a distance between the radiation detection unit and the examinee by being placed on the placement unit.

According to the inventions described in claims 1 and 6, it is possible to diagnose the entire breast of the examinee while the breast is in the free drooping state, regardless of a drooping state of the examinee's breast by adjusting the distance between the radiation detecting unit and the examinee.

According to the invention described in claim 2, it is easy to carry the distance adjustment member and it is possible to flexibly deal with a difference in a physique of the examinee by adjusting an installation position with respect to the placement unit.

According to the invention described in claim 3, it is possible to prevent positional deviation of each part by the connection member.

According to the invention described in claim 4, it is possible to fix and position the part configured to place the head thereon, on the placement unit.

According to the invention described in claim 5, it is not necessary to strictly position the distance adjustment member with respect to the placement unit, and the inspection can be easily performed.

### BRIEF DESCRIPTION OF THE DRAWINGS

FIG. 1 is a perspective view of a nuclear medicine diagnostic apparatus for breast examination to which the present invention is applied;

FIG. 2 is a schematic side view illustrating a state in which an examination of an examinee M is executed by a nuclear medicine diagnostic apparatus for breast examination;

FIG. 3 is a schematic side view illustrating a state in which an examination of an examinee M with her breast largely drooped is executed by a nuclear medicine diagnostic apparatus for breast examination;

FIG. 4 is a perspective view of a distance adjustment member 10;

FIG. 5 is a side view of the distance adjustment member 10;

FIG. 6 is a schematic diagram illustrating a state in which a distance adjustment member 10 is placed on a placement unit 1 and an examination of the examinee M is executed; and

FIG. 7 is a schematic side view illustrating a state in which the distance adjustment member 10 is placed on the placement unit 1 and an examination of the examinee M is executed.

### DETAILED DESCRIPTION

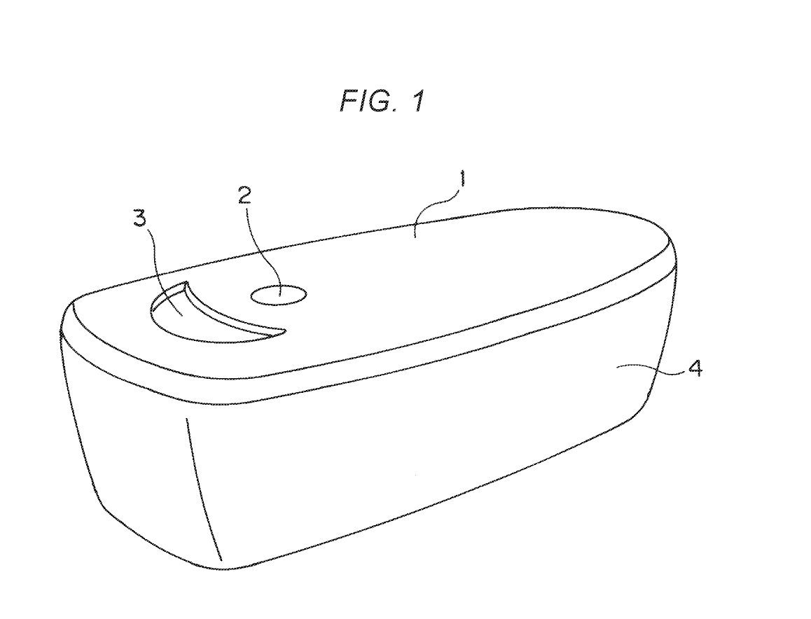

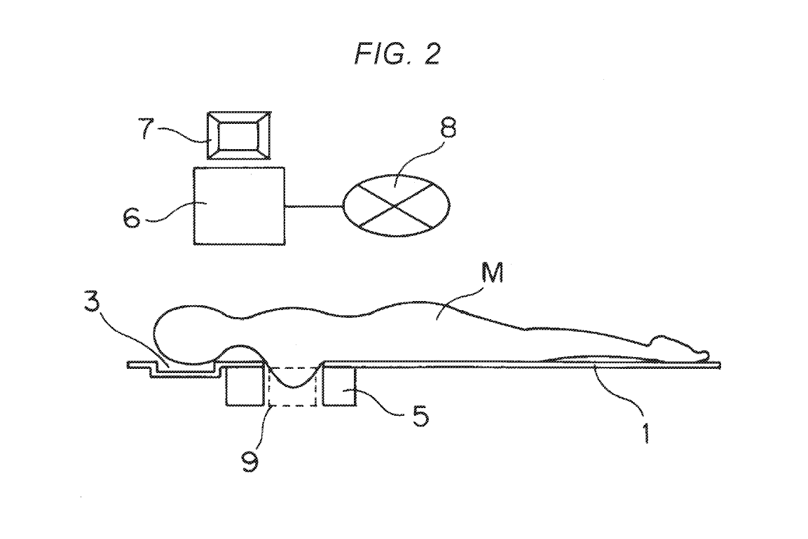

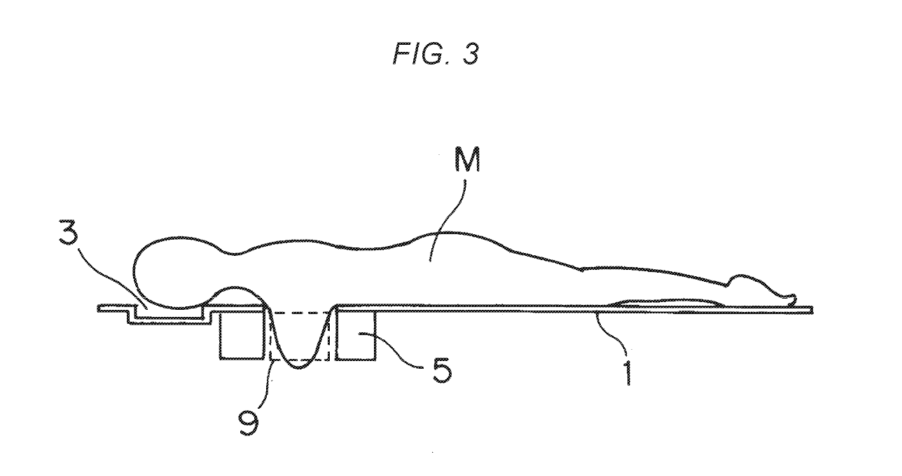

Hereinafter, embodiments of the present invention will be described with reference to the drawings. FIG. 1 is a perspective view of a nuclear medicine diagnostic apparatus for breast examination to which the present invention is applied, and FIG. 2 is a schematic side view illustrating a state in which an examination of an examinee M is executed by the nuclear medicine diagnostic apparatus for breast examination. Further, FIG. 3 is a schematic side view illustrating a state in which an examination of an examinee M with her breast largely drooped is executed by the nuclear medicine diagnostic apparatus for breast examination.

The nuclear medicine diagnostic apparatus for breast examination, for example, is called a PET apparatus for a Mammo, and has a configuration in which a placement unit 1 for placing the examinee M thereon is disposed on a main body 4. A concave portion 3 on which a head of the examinee M is placed and an opening 2 through which the examinee M's breast droop are formed in the placement unit 1. In a state where the examinee M is in a prone position on the placement unit 1, a radiation detection unit 5 is disposed in a ring-like shape around the examinee M′ breast drooping from the opening 2 of the placement unit 1.

In this nuclear medicine diagnostic apparatus for breast examination, the breast of the examinee M to whom a radiopharmaceutical has been administered in advance is inserted into a ring-shaped radiation detection unit 5 in which a tunnel-like examination space is formed, and pair annihilation photons of 511 keV emitted from the breast of the examinee M are detected by the radiation detection unit 5. Then, a time at which the photons are detected is measured, and when a difference between the times detected by two detectors in the radiation detection unit 5 is within a certain time, the photons are counted as a pair of pair annihilation photons and, furthermore, specified as being at positions on a straight line of a pair of the detectors that detected a pair annihilation occurrence point. Emission data thus obtained is accumulated in a console 6 and image reconstruction is performed using the emission data accumulated in the console 6 to acquire a PET image. Then, the PET image thus acquired is displayed on a monitor 7 and transferred to a server in a hospital via an intra-hospital network 8.

At this time, the breast of the examinee M needs to be disposed within a detection range 9 of radiation in the radiation detection unit 5 in a free drooping state. However, the breast of the examinee M may not fit within the radiation detection range 9 of the radiation detection unit 5 as illustrated in FIG. 3, due to drooping of the breast depending on a physique and an age of the examinee M. In such a case, since the entire area of the breast of the examinee M cannot be examined, it is not impossible to properly execute a nuclear medicine diagnosis. Therefore, in the nuclear medicine diagnostic apparatus for breast examination according to the present invention, a distance adjustment member 10 is used.

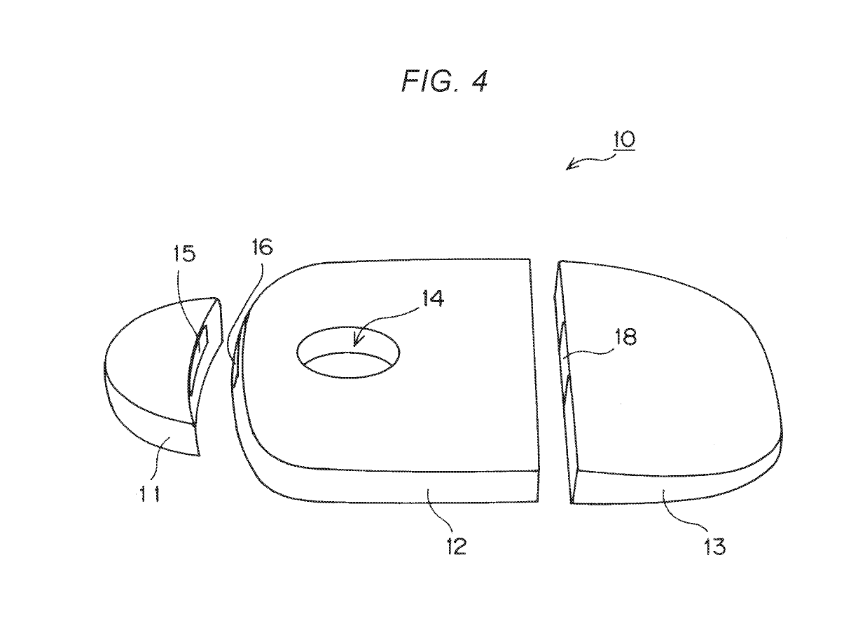

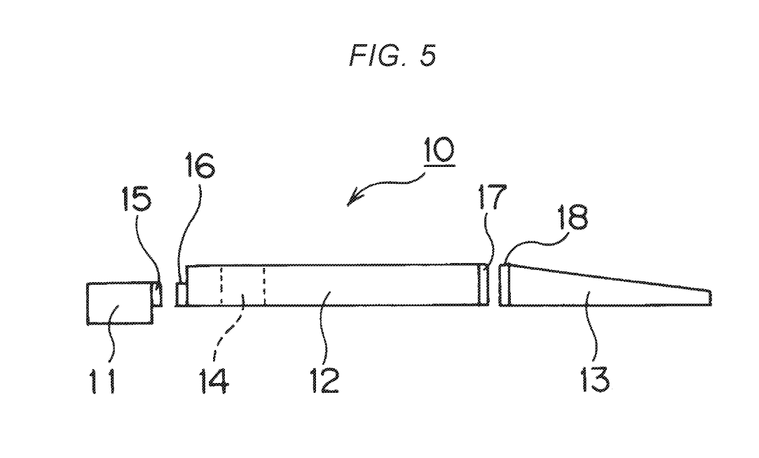

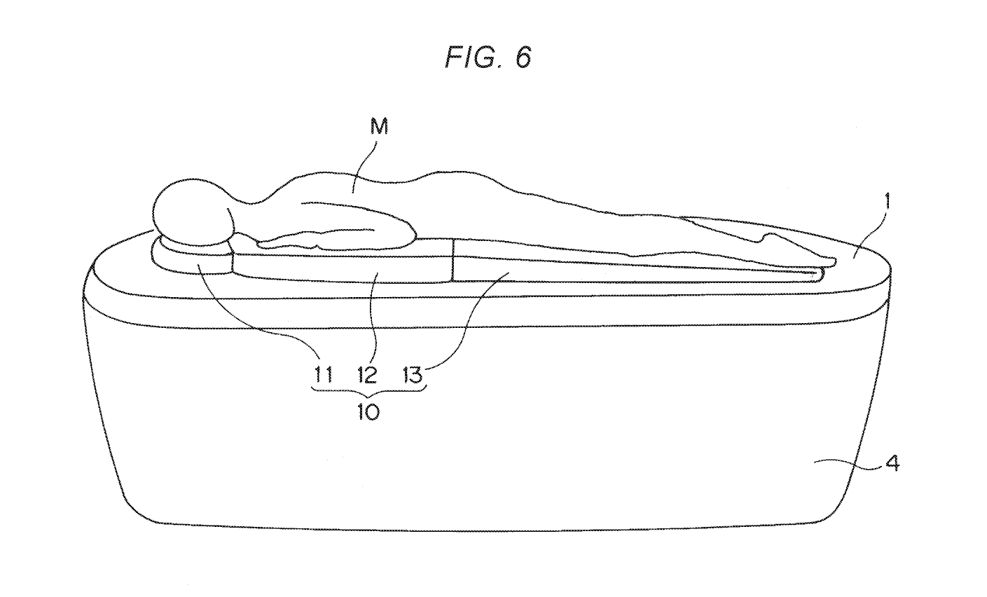

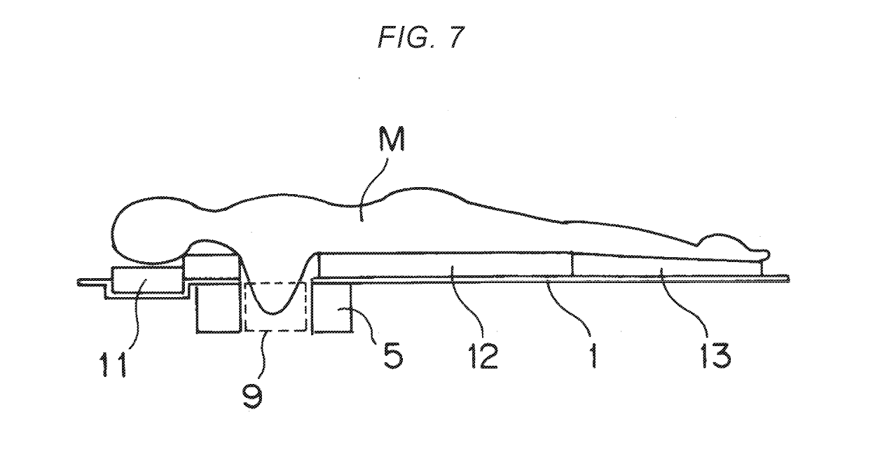

FIG. 4 is a perspective view of a distance adjustment member 10. FIG. 5 is a side view of the distance adjustment member 10. FIG. 6 is a schematic diagram illustrating a state in which a distance adjustment member 10 is placed on a placement unit 1 and an examination of the examinee M is executed. FIG. 7 is a schematic side view illustrating a state in which the distance adjustment member 10 is placed on the placement unit 1 and an examination of the examinee M is executed.

The distance adjustment member 10 according to the present invention includes a first part 11, a second part 12 and a third part 13. The first part 11 is a part on which a head of the examinee M is placed. The second part 12 is mainly a part on which a body region of the examinee M is placed. The third part 13 is a part on which legs of the examinee M are placed. These first part 11, second part 12 and third part 13 can be connected and divided with each other by action of surface fasteners 15, 16, 17, 18 functioning as connecting members according to the present invention.

Among the first part 11, the second part 12 and the third part 13 constituting the distance adjustment member 10, the first part 11 for placing the head of the examinee M thereon may be engaged with a concave portion 3 provided on the placement unit 1. Further, an opening 14 through which the breast of the examinee M is to droop is formed in the second part 12 on which the body region and the like of the examinee M is placed. An inner diameter of the opening 14 is larger than an inner diameter of the opening 2 formed in the placement unit 1. Further, a surface of the third part 13 is inclined.

The first part 11, the second part 12, and the third part 13 constituting the distance adjustment member 10 have a configuration of being lightweight and soft, for example, a configuration in which a periphery of urethane is covered with synthetic leather. Therefore, it is easy to install and move the first part 11, the second part 12, and the third part 13 constituting the distance adjustment member 10, and there is no fear of injury even though the parts are mistakenly dropped on a foot and the like of the examinee M or an operator. Further, since the first part 11, the second part 12, and the third part 13 constituting the distance adjustment member 10 may be connected to and divided from each other freely, it is possible to install and move the parts easily by setting the parts in a divided state.

The first part 11, the second part 12, and the third part 13 constituting the distance adjustment member 10 have a hardness of, for example 60 newtons or greater so as not to be excessively deformed even when the examinee M having a large physique is placed on the parts. Further, at least lower faces of the first part 11, the second part 12, and the third part 13 constituting the distance adjustment member 10 have a large friction coefficient against the placement unit 1. Therefore, movement of the first part 11, the second part 12, and the third part 13 on the placement unit 1 may be suppressed.

When the breast of the examinee M does not fit within the radiation detection range 9 of the radiation detection unit 5 as illustrated in FIG. 3, the distance adjustment member 10 is placed on the placement unit 1, and the examinee M is placed in a prone position on the distance adjustment member 10 as illustrated in FIG. 7. Then, the breast of the examinee M is caused to droop to the radiation detection range 9 in the radiation detection unit 5 via the opening 14 formed in the second part 12.

Thus, in the state illustrated in FIG. 3, it is possible to examine a region of the breast of the examinee M, which is not located within the radiation detection range 9 in the radiation detection unit 5. Therefore, the entire region of the breast of the examinee M can be examined while the breast is in a free drooping state, by executing the examination both in the state where the distance adjustment member 10 is not used as illustrated in FIG. 3 and in the state where the distance adjustment member 10 is used as illustrated in FIG. 7.

At this time, since there is no need to move the radiation detection unit 5 and the placement unit 1, it is possible to simplify a configuration of the nuclear medicine diagnostic apparatus for breast examination, and furthermore, it is possible to eliminate a possibility of collision with the examinee M accompanying the movement of the radiation detection unit 5 or the placement unit 1 or a possibility of being clamped by a member that moves a part of a body of the examinee M.

Here, as described above, the inner diameter of the opening 14 formed in the second part 12 of the distance adjustment member 10 is larger than the inner diameter of the opening 2 formed in the placement unit 1. Therefore, when the second part 12 is placed on the placement unit 1, it is good as long as the opening 2 formed in the placement unit 1 is disposed approximately in the vicinity of a center of the opening 14 formed in the second part 12 of the distance adjustment member 10. Therefore, it is unnecessary to strictly position the second part 12 with respect to the placement unit 1, and the examination can be performed easily.

Further, the first part 11 of the distance adjustment member 10 may be engaged with the concave portion 3 formed in the placement unit 1, and the first part 11, the second part 12 and the third part 13 are configured to be connected to each other by action of the surface fasteners 15, 16, 17 and 18. Therefore, the first part 11, the second part 12 and the third part 13 constituting the distance adjustment member 10 may be fixed easily.

Further, when a height of the examinee M is large, the height of the examinee M may be dealt with by arranging the first part 11, the second part 12, and the third part 13 constituting the distance adjustment member 10 at positions separated from each other on the placement unit 1. Even in a case where the first part 11, the second part 12 and the third part 13 are arranged to be spaced apart from each other, the movement of the first part 11, the second part 12, and the third part 13 on the placement unit 1 may be suppressed by setting the lower faces of the first part 11, the second part 12 and the third part 13 constituting the distance adjustment member 10 to have a large friction coefficient against the placement unit 1.

Further, it is necessary to set a thickness of the second part 12 of the distance adjustment member 10 so that the entire region of the breast of the examinee M may be examined, by executing the examination both in the state where the distance adjustment member 10 is not used as illustrated in FIG. 3 and in the state where the distance adjustment member 10 is used as illustrated in FIG. 7. This thickness is, for example, about 7 cm. At this time, in order to deal with a case where the drooping state of the breast of the examinee M changes greatly depending on the examinee M, a plurality of second parts 12 with different thicknesses may be prepared.

1. A nuclear medicine diagnostic apparatus for breast examination comprising:
a placement unit where an opening is formed and a radiation detecting unit disposed around an examinee's breast drooping from the opening of the placement unit in a state where the examinee is in a prone position on the placement unit, wherein the nuclear medicine diagnostic apparatus comprises a distance adjustment member that has an opening connected to the opening of the placement unit when the distance adjustment member is placed on the placement unit, and changes a distance between the radiation detection unit and the examinee by being placed on the placement unit.

2. The nuclear medicine diagnostic apparatus for breast examination according to claim 1, wherein the distance adjustment member is divided into a plurality of parts with respect to a direction of a body axis of the examinee.

3. The nuclear medicine diagnostic apparatus for breast examination according to claim 2, comprising a connection member that connects each part of the distance adjustment member to each other.

4. The nuclear medicine diagnostic apparatus for breast examination according to claim 3, wherein one of the parts of the distance adjustment member is configured to place a head of the examinee thereon, and
wherein the part configured to place the head of the examinee thereon is engageable with a concave portion formed in the placement unit.

5. The nuclear medicine diagnostic apparatus for breast examination according to claim 1, wherein an inner diameter of the opening formed in the distance adjustment member is larger than an inner diameter of the opening formed in the placement unit.

6. An adjustment apparatus that is used in a nuclear medicine diagnostic apparatus for breast examination including a placement unit where an opening is formed and a radiation detecting unit disposed around an examinee's breast drooping from the opening of the placement unit in a state where the examinee is in a prone position on the placement unit, wherein the adjustment apparatus has an opening connected to the opening of the placement unit when the adjustment apparatus is placed on the placement unit, and changes a distance between the radiation detection unit and the examinee by being placeable on the placement unit.

7. The nuclear medicine diagnostic apparatus for breast examination according to claim 2, wherein an inner diameter of the opening formed in the distance adjustment member is larger than an inner diameter of the opening formed in the placement unit.

8. The nuclear medicine diagnostic apparatus for breast examination according to claim 3, wherein an inner diameter of the opening formed in the distance adjustment member is larger than an inner diameter of the opening formed in the placement unit.

9. The nuclear medicine diagnostic apparatus for breast examination according to claim 4, wherein an inner diameter of the opening formed in the distance adjustment member is larger than an inner diameter of the opening formed in the placement unit.
